(19) United States
(12) Patent Application Publication
(10) Pub. No.: US 2008/0087740 A1
(43) Pub. Date: Apr. 17, 2008

(54) Air Treatment Device

(75) Inventors: Daniel C. Gusenoff, Newton, MA (US); Alyssa I. Dubin, Newton, MA (US)

Correspondence Address: BROWN, RUDNICK, BERLACK & ISRAELS, LLP., BOX IP, 18TH FLOOR, ONE FINANCIAL CENTER, BOSTON, MA, 02111, US

(73) Assignee: JAPAN SCIENCE AND TECHNOLOGY AGENCY, KAWAGUCHI-XHI, KAITAMA JAPAN (JP)

(21) Appl. No.: 11/596,737

(22) PCT Filed: Oct. 6, 2005
(86) PCT No.: PCT/US05/35992

§ 371 Date: Nov. 16, 2006

Related U.S. Application Data
(60) Provisional application No. 60/622,833, filed on Oct. 28, 2004.
(60) Provisional application No. 60/624,476, filed on Nov. 1, 2004.

(30) Foreign Application Priority Data
Jan. 26, 2005 (US) 11045720

(51) Int. Cl. A61L 9/04

(52) U.S. Cl. 239/55

(57) ABSTRACT

An air treatment device for use with a refuse or non-refuse container or receptacle is provided. The air treatment device includes a cover member and housing member for housing an air treating material. The housing and the cover member include an air treating material therebetween. The housing is rotatably and removably or pivotally coupled to the cover member and is deformable with respect to the cover. The housing is continuously under tension and biased toward or against the air treating material so as to provide continuous contact of the air treating material and the housing. The housing and the cover component include retaining devices for removably or pivotally coupling to one another. The air treatment device further includes an indicator device for indicating a period of time. The indicator device may be integrally molded or removably coupled to the cover component of the air treatment device.

### CROSS-REFERENCE TO RELATED APPLICATIONS

This application claims priority to U.S. Provisional Patent Application Ser. No. 60/622,833, filed Oct. 28, 2004, U.S. Utility patent application Ser. No. 11/045,720, filed Jan. 26, 2005 and U.S. Provisional Patent Application Ser. No. 60/622,447, filed Mar. 16, 2205, the entire contents of which are incorporated herein by reference.

### BACKGROUND

1. Technical Field

The present disclosure relates generally to air treatment devices, and more particularly, to air treatment devices for use with refuse or non-refuse containers and receptacles.

2. Background of the Related Art

Containers and receptacles of various shapes, sizes and configurations are utilized in kitchens, garages, bathrooms, nurseries, workrooms, professional offices, hotels, resorts, health clubs, health care facilities, theaters, amusement parks, garbage collection areas and the like. Odors and germs gather in these containers and receptacles depending upon the waste or matter contained therein. Additionally, odors emanating from such containers and receptacles oftentimes attract insects or rodents.

Devices disseminating various kinds of air treating materials, such as air freshening, odor absorbing, disinfectant, insecticidal and pesticide materials have been developed to solve these problems. Common solid air treatment devices include, but are not limited to, (i) disks, domes, dishes or other dispensers which hold air treating blocks or cakes; (ii) disks, domes, dishes or other dispensers which hold gel material which, when it dries or shrinks, releases a vaporized air-treating composition into the air, (iii) disks, domes, dishes or other dispensers which hold paperboard coated with a vaporizable composition, (iv) disks, domes, dishes or other dispensers which hold scented disks, and (v) disks, domes, dishes, bags or other dispensers which hold liquid solutions, and the like. Common deodorizing, odor absorbing, disinfecting, antibacterial or insecticidal or pesticide solid air treating materials include, but are not limited to, blocks or cakes, gels, paperboard, scented disks, bags or other means for holding liquid solutions, and the like.

Most solid air treatment devices have inferior construction in that they only hold one particular size, shape or configuration of a solid air treating material and do not permit users to purchase a variety of solid air treating material to be used in that same air treatment device. Also, most solid air treatment devices, after a period of time, result in dried or shrunken material that remains within the air treatment device. Most dried or shrunken solid air treating material shakes, rattles or rolls when the solid air treatment device is moved or affixed to a surface which moves or is moved. Moreover, most solid air treatment devices do not affix very well or are too heavy to be mounted on the underside of a refuse or non-refuse container's or receptacle's lid or cover. The currently available devices do not provide for sufficient air-flow to maximize the effectiveness of the solid air treating material. Additionally, current solid air treatment devices generally fail to notify the user as to when the effectiveness of a solid air treating material is no longer effective.

Thus, an object of the present disclosure is to provide an air treatment device including a solid air treating material dispenser that is configured with a universal, flexible holder mechanism that is constantly under tension, which may be configured onto the underside of any sized or shaped lid or cover of any sized or shaped refuse or non-refuse container or receptacle, and that is able to hold any size, shape or configuration of solid air treating material.

It is another object of the present disclosure to provide an improved air treatment device that keeps any sized or shaped solid air treating material securely placed when installed onto the underside of a refuse or non-refuse container's or receptacle's lid or cover, even upon the eventual drying or shrinking of the air treating material.

It is another object of the present disclosure to have multiple means of affixing the air treatment device to the underside of a refuse or non-refuse container's or receptacles lid or cover.

It is another object of the present disclosure to have multiple large perforations to enable increased air-flow through the air treatment device.

It is another object of the present disclosure to be made of plastic, rubber, metal, steel, wood or any other material.

It is another object of the present disclosure to have a device for indicating the depletion of the air treating material contained within the air treatment device.

Other objects and advantages of the present invention shall become apparent from the accompanying description and drawings.

### SUMMARY

Accordingly, air treatment devices for use with refuse or non-refuse containers or receptacles are disclosed. The air treatment devices include a cover member and housing member for housing an air treating material. The housing and the cover member include an air treating material therebetween. The housing is rotatably and removably or pivotally hinged, clasped, retained or otherwise coupled to the cover member and is deformable with respect to the cover. The housing, which is constantly under tension, is biased toward or against the air treating material so as to provide continuous contact of the air treating material and the housing as the air treating material shrinks or evaporates over time. The housing and the cover component include retaining devices for removably, pivotally, claspedly, retainingly or othewisedly coupling one to another. The air treatment device further includes an indicator device for indicating a period of time. The indicator device may be coupled or removably coupled to the cover component of the air treatment device.

Additionally, an air treatment system including a housing, which is constantly under tension, for securing an air treating material and a retaining device for removably, pivotally, claspedly, retainingly integradedly or otherwisedly coupling the housing to a refuse or non-refuse container or receptacle by hinges, clasps, retention means or otherwise is provided. The housing is removably pivotally, claspedly, retainingly or otherwisedly coupled to a cover portion of the refuse or non refuse container or receptacle by hinges, clasps, retention means or otherwise. The housing includes a biased portion, which is constantly under tension, for securely retaining the air treating material, wherein the biased portion retains a constant bias against the air treating material. The air treatment system further includes an indicating device for indicating a time frame and a plurality of through holes for allowing air to contact the air treating material.

Moreover, an air treatment device including a removably pivotally, claspedly, retainingly or otherwisedly mountable basket and cover coupled to the basket is provided by hinges clasps, retention means or otherwise. The basket is deformable and under tension with respect to the cover and is biased toward the cover. The cover and basket include respective coupling members, such as flanges, screw threads, rails, detents, projections, tangs, living hinges, spring biased hinges and the like. The air treatment device further includes an attachment device for attaching the cover to a refuse or non-refuse container or receptacle. The attachment device may include hook and loop fasteners, fastening clips, tape, adhesive, magnets, suction cups, screws, nails, other hardware and the like.

Objects and advantages of the present disclosure are set forth in part herein and in part will be obvious therefrom, or may be learned by practice of the present disclosure which is realized and attained by the instrumentalities and combinations pointed out in the appended claims for the devices and methods of the present disclosure consisting of its constituent parts, constructions, arrangements, combinations, steps and improvements herein shown and described.

### BRIEF DESCRIPTION OF THE DRAWINGS

The objects and features of the present disclosure are set forth with particularity in the appended claims. The present disclosure, as to its organization and manner of operation, together with further objectives and advantages may be understood by reference to the following description, taken in connection with the accompanying drawings, in which:

FIG. 1 is a plan view of an air treatment device in combination with a receptacle in accordance with the present disclosure;

FIGS. 2A, 2B and 2C are plan views of the air treatment device of FIG. 1;

FIG. 2D is an enlarged view of FIG. 2A depicting a portion of the air treatment device and freshness indicator in accordance with the present disclosure;

FIG. 2E is an enlarged view of the underside of FIG. 2D in accordance with the present disclosure.

FIG. 3 is an exploded view of an air treatment device in accordance with the present disclosure;

FIG. 4 is a perspective view of an air treatment device in accordance with the present disclosure;

FIG. 5 is a perspective view of a retention ring of the air treatment device in accordance with an embodiment of the present disclosure;

FIG. 6A is a perspective view of a retaining device of the air treatment device in accordance with an embodiment of the present disclosure;

FIG. 6B is a side view of the retaining device of FIG. 6A;

FIG. 6C is a side view of the retaining device of FIG. 6A in a retaining position with an original size of air treating material;

FIG. 6D is a side view of the retaining device of FIG. 6A in a retaining position with a dried, shrunken or partially evaporated air treating material.

FIGS. 7A, 7B and 7C are plan views of an alternate embodiment of an air treatment device in accordance with the present disclosure;

FIG. 8 is an exploded view of the air treatment device of FIGS. 7A, 7B and 7C;

FIG. 9 is a perspective view of the air treatment device of FIGS. 7A, 7B and 7C;

FIG. 10A is a perspective view of an alternate embodiment of the air treatment device in accordance with the present disclosure, showing a hinged retaining device in an open extended position;

FIG. 10B is a perspective view of an alternate embodiment of the air treatment device in accordance with the present disclosure, showing a hinged retaining device in a closed position;

FIG. 10C is a perspective view of an alternate embodiment of the air treatment device in accordance with the present disclosure, showing a hinged retaining device in an open extended position; and

FIG. 10D is a perspective view of an alternate embodiment of the air treatment device in accordance with the present disclosure, showing a hinged retaining device in a closed position.

### DETAILED DESCRIPTION OF EXEMPLARY EMBODIMENTS

The exemplary embodiments of the air treatment devices disclosed are discussed in terms of air treatment devices for use with receptacles and containers including, for example, refuse or non-refuse receptacles and containers. The presently disclosed air treatment devices are contemplated for use as an integral cover or non-integral lid portion of a container or receptacle as well as a portable or stand alone unit for incorporation with a container or receptacle. It is contemplated that the air treatment device of the present disclosure may be employed with, for example, trash barrels as part of a trash barrel lid or cover or a separate unit for attachment to the underside of a trash barrel lid.

In the discussion that follows, the term trash barrel refers to any type of refuse or non-refuse container or receptacle that is used to collect, store or house waste, trash, food or any other matter and the term cover refers to a cover or lid portion that is used to shield or cover the top open portion of a trash barrel. As used herein, the term “top” generally refers to a portion of the trash barrel or cover that faces or is directed upward (e.g., upward toward the ceiling), while the term “bottom” generally refers to a portion of the trash barrel or cover that faces or is directed downward (e.g., downward toward the floor).

The following discussion includes a description of an air treatment device used as a trash barrel cover and separate air treatment device for use with a cover or trash barrel. Reference will now be made in detail to the exemplary embodiments of the disclosure, which are illustrated in the accompanying Figures.

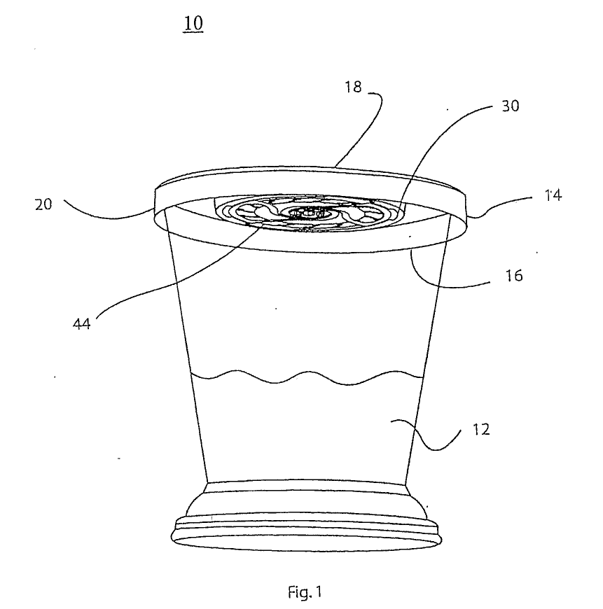

Turning now to the Figures, wherein like components are designated by like reference numerals throughout the several views. Referring to FIG. 1, there is illustrated a combination air treatment device and refuse receptacle 10 in accordance with the principles of the present disclosure. Air treatment device and refuse receptacle 10 includes a trash barrel 12, for example, a circular trash barrel having a closed bottom end and open top end for the receiving of refuse or non-refuse waste, trash or other matter. It is contemplated within the present disclosure that trash barrel 12 may include any typical trash barrel for use in home, office or industrial uses and may be of any size, shape or configuration as is well known. An air treatment device 14 may act as a cover for trash barrel 12 or as a separate attachment member to a cover of a trash barrel 12. Whether acting as a cover of a trash barrel or as an attachment to a cover for a trash barrel, it is contemplated within the present disclosure that the air treatment device 14 may include any size, shape or configuration that can be employed to cover a trash barrel 12 or attach to a cover of a trash barrel.

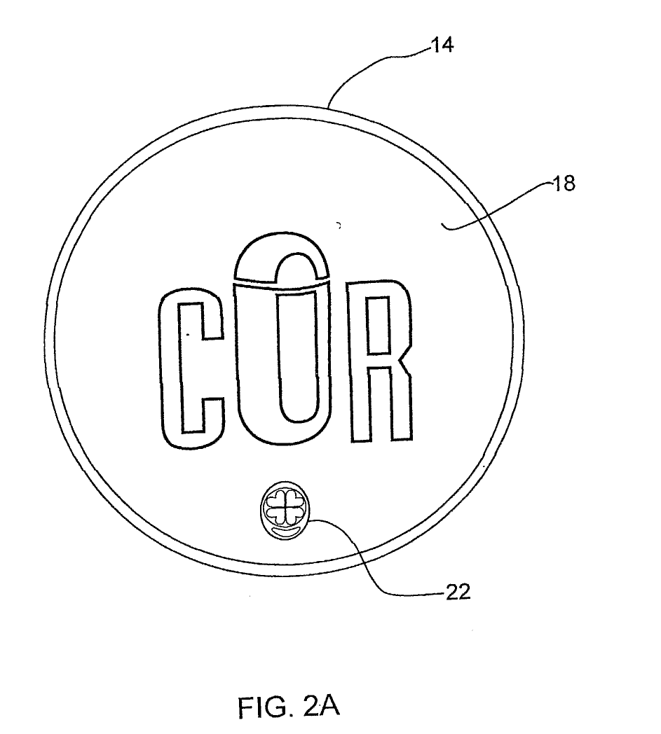

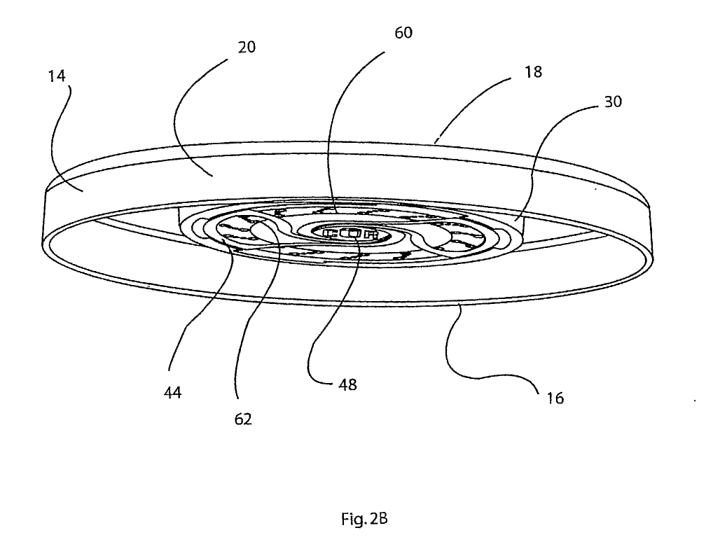

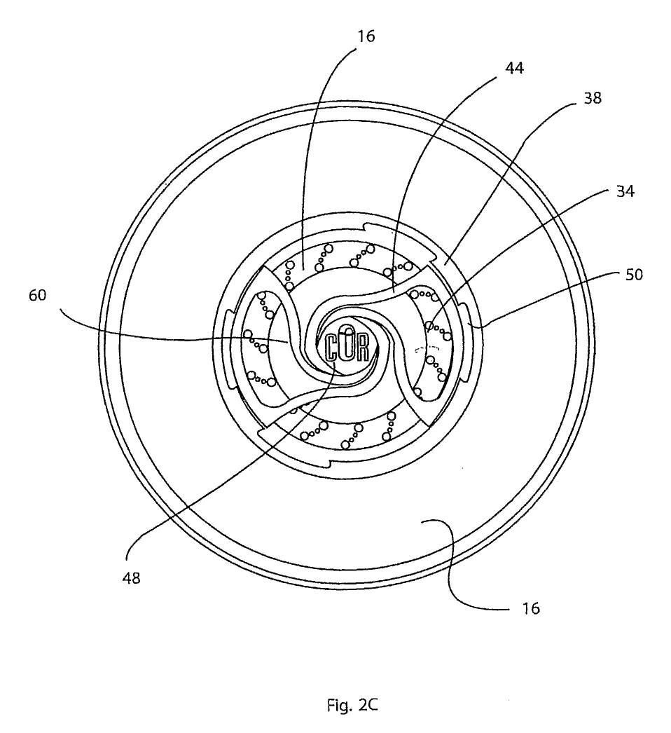

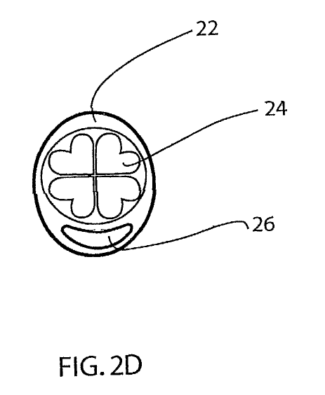

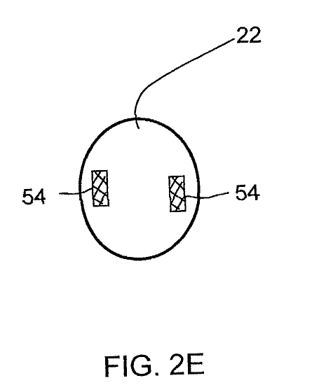

With reference to FIGS. 1, 2A, 2B and 2C, the air treatment device 14 includes a top 18, bottom 16 and sidewall 20 portion and may be configured as a separate stand alone, hinged or like member that fits onto or over the open end of trash barrel 12. Top portion 18 of the air treatment device 14 may include an air freshness indicator means 22 (as described below) for use by a consumer in determining a period of freshness or time. As will be discussed in detail below, bottom portion 16 is designed to incorporate the air treatment device in accordance of the present disclosure.

The air treatment device 14 may be formed of any size, shape or configuration (e.g., oval, circular, square, rectangular, diamond, octagonal or the like) and be manufactured from any suitable material including, for instance, plastic, rubber, metal, steel, wood or the like. The retaining device 44 is constantly under tension and preferably perforated with multiple openings in any size, shape or configuration (e.g., tear drops, rectangles, ovals, circles, squares, diamonds, octagons, zig zag patterns or the like) for allowing appropriate air flow to contact air treating material 60. The air treating material 60 may include an air deodorizer component, an air freshener component, an odor absorbing component which may consist of carbon, carbon crystals or other chemicals as is known in the art of odor absorbing materials, a disinfectant component, an insecticidal component, a pesticide component, an antibacterial component or the like as is known in the air treating material art, with or without fragrance, of any size, shape, or configuration (e.g., oval, circular, square, rectangular, diamond, octagonal, or the like) that is capable of being housed between the bottom portion 16 and the retaining member 44. Moreover, air treating material 60 may be formed as a solid, for example a block, cake, gel, paperboard scented disk or otherwise as is known in the air treating material art, or liquid, for example, a liquid air treating material that is housed by a container or bag as is known in the air treating material art. Any portion of air treatment device 14 including the top 18, bottom 16 and sidewall 20 may include perforations for allowing air flow.

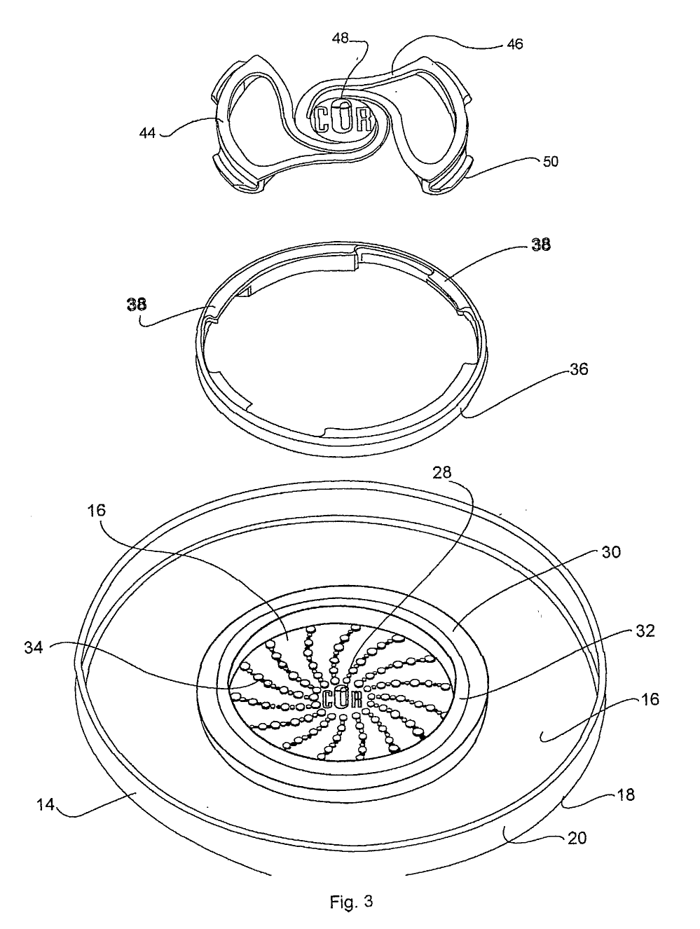

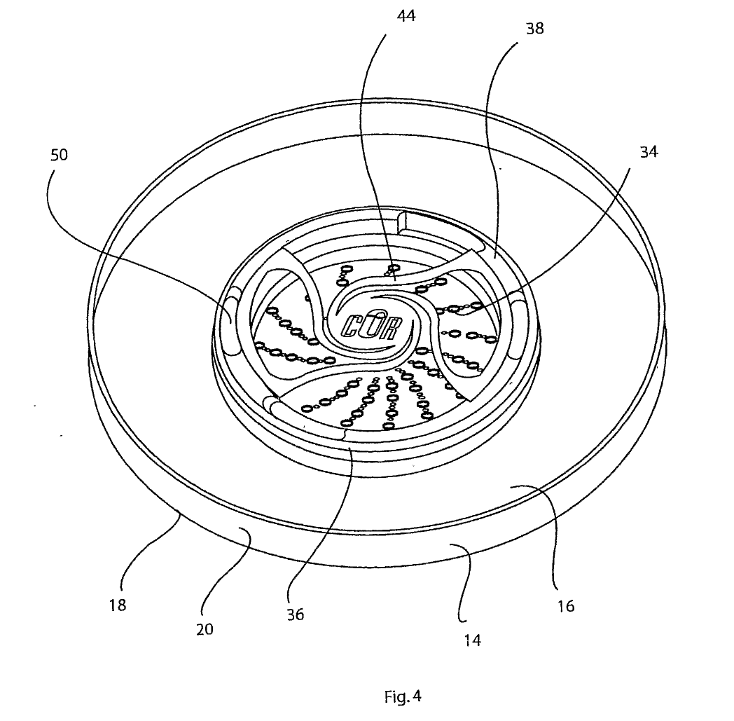

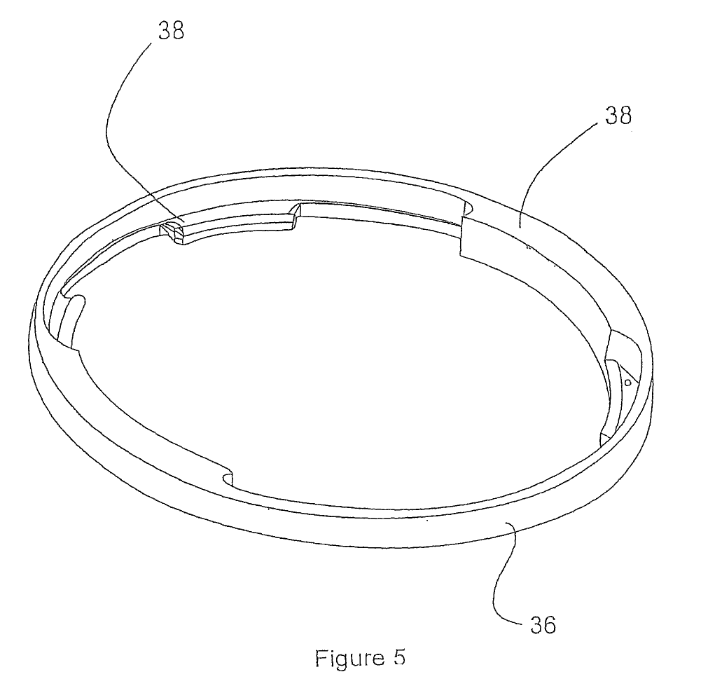

Now referring to FIG. 3, air treatment device 14 is shown in an upside down exploded fashion depicting various portions thereof. More particularly, bottom portion 16 of the air treatment device 14 is bounded by sidewall 20. Sidewall 20 is designed to fit over or onto a rim portion (not shown) of trash barrel 12. Bottom portion 16 of the air treatment device 14 includes a flange or retaining member 30 integrally formed thereon. Flange 30 may include retaining ridge or screw thread 32 that is configured for attachment to a retention ring 36. As best seen in FIG. 5, retention ring 36 includes flange or screw thread 38 for cooperative attachment to retaining ridge or screw thread 32 of flange 30 within bottom portion 16 of the air treatment device 14. Alternatively, and as best shown in FIG. 4, flange 30 may be replaced by retention ring 36 that is integrally molded to bottom portion 16 of the air treatment device 14. Retention ring 36 includes flange or screw thread 38 for cooperative attachment to a retaining ridge or screw thread 50 of a retaining device or basket 44 (to be discussed below). Referring back to FIGS. 3 and 4, bottom portion 16 of the air treatment device 14 includes a center portion 28 for placement of air treating material 60 (to be discussed below). Center portion 28 includes a plurality of nubs, nodules or bumps 34 protruding (in the bottom direction of the trash barrel 12) from the bottom portion 16. These nubs may be of varying heights, shapes, sizes and configurations and are configured for allowing air flow between the air treating material 60 and the bottom portion 16 of the air treatment device 14.

With reference to FIGS. 3, 4, 6A and 10B, the basket or retaining device 44 includes a plurality of arm members 46 connected to center portion 48. Retaining device 44 includes retaining means 50 in the form of flanges, screw threads, detents hinges, clasps or the like. Retaining means 50 may include a hinged component where a portion of the retaining device 44 is hingedly connected to the retaining ring 36 or bottom portion 16 of the air treatment device 14. Retaining means 50 includes receiving surface 52 for receiving flange or screw thread 38 of retention ring 36. It is contemplated herein that the cooperating elements of retention ring 36 and retaining device 44 may include screw threads, flanges, detents, clips or other removably or pivotally or hinged or clasped or otherwise retained retaining elements that are configured for the removal or pivoting of the retaining device 44 from the retention ring 36. Retaining device 44 may be formed as any shaped housing or basket having one or more air holes or pathways 62 (e.g., holes, slits, mesh and the like) for allowing increased or decreased air flow to the air treating material 60 housed therein.

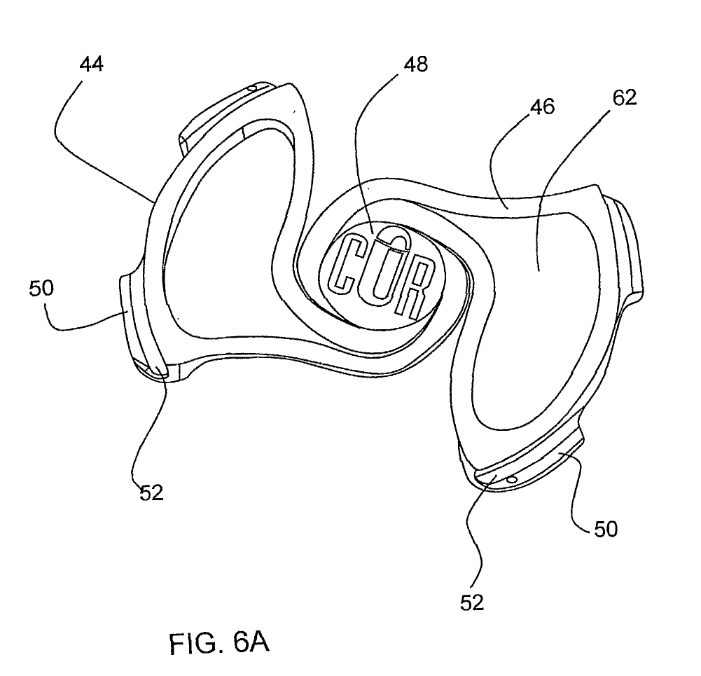

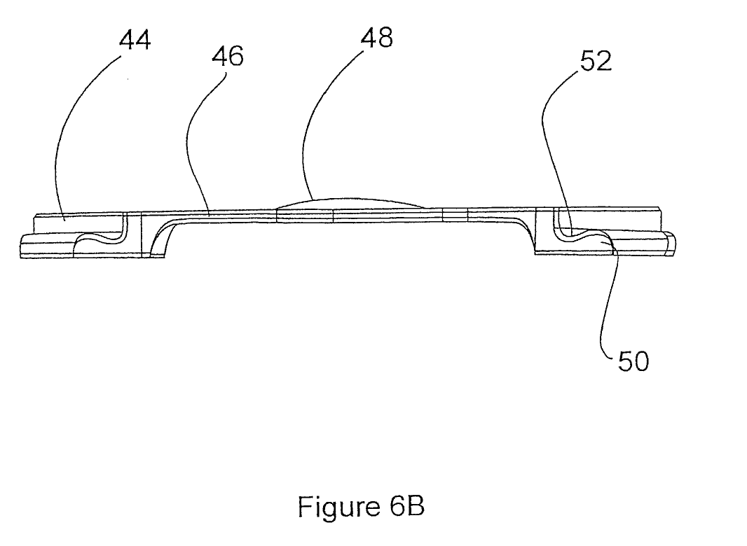

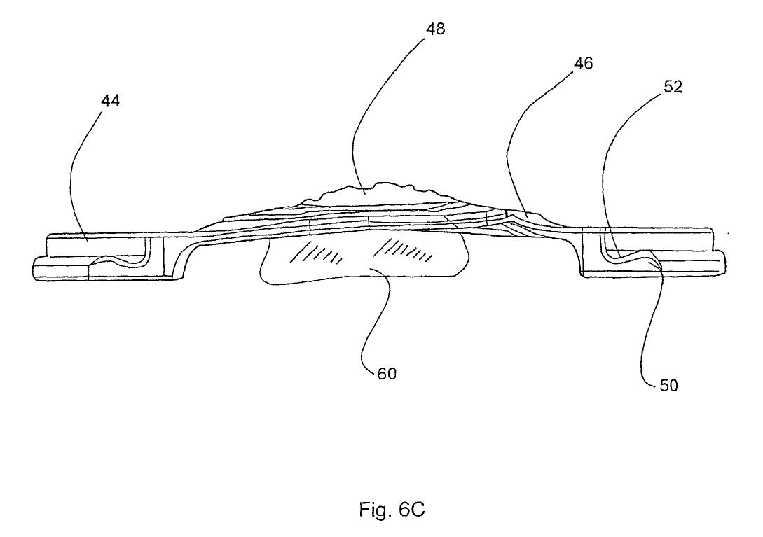

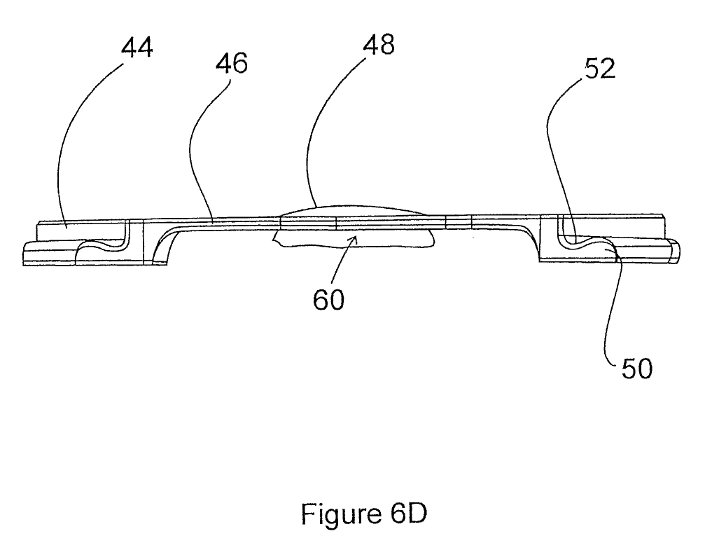

Referring to FIGS. 4, 6A, 6B, 6C, 6D and 10A, retaining device 44 is configured to be removably or pivotally or hinged or clasped or otherwise mounted to bottom portion 16 of the air treatment device 14 (via retention ring 36). Retaining device 44 is configured to be under continuous tension and to supply a consistent bias in the upward or downward direction or against to the air treating material 60 housed between the retaining device 44 and bottom portion 16 of the air treatment device 14 (FIG. 6C). More specifically, retaining device 44 and/or arms 46 and center portion 48 of retaining device 44 are formed from materials that allow a measure of tension and flexibility and elastic or biased contraction in an upward direction (i.e., toward the bottom portion 16 of the air treatment device 14) or downward direction (i.e. toward the bottom portion of trash barrel 12). It is contemplated within the scope of the present disclosure that retaining device 44 is constantly under tension and may deform, expand or be shaped to accommodate any sized or shaped air treating material 60. For example, the retaining device may take the shape of a basket or flexible housing. Retaining device 44 may include a plurality of the air holes 62 and may be formed from any material providing elastic or biasing functionality such as silicone or rubber based plastics, mesh, netting or other material as is well known in the art. It is contemplated that retaining device 44 may further include elastic enhancing devices such as springs (e.g., leaf springs), bungee cords, rubber bands and the like as is well know in the art located along or as part of arms 46 and/or center portion 48.

In operation and by way of non-limiting example, upon placement of the air treating material 60 (e.g., a solid of a particular size, shape, or configuration) between the bottom portion 16 and retaining device 44, the retaining device 44 would be under tension and biased in an upward direction toward the top portion 18 and thereby continually secures the air treating material 60 between the bottom portion 16 and retaining device 44 components. Upon the gradual evaporation and shrinking of the air treating material 60, the retaining device 44 would continue to be under tension and be biased toward the top portion 18 and continue to provide a secure fit of the air treating material 60. An additional non-limiting operational example is that upon placement of the air treating material 60 (e.g., a solid of a particular size, shape, or configuration) between the bottom portion 16 and retaining device 44, the retaining device 44 would be biased in a downward direction, for example, toward the bottom of the trash barrel 12 (FIG. 1) and thereby continually secures the air treating material 60 between the bottom portion of 16 and retaining device 44 components. Upon the gradual evaporation and/or shrinking of the air treating material 60, the retaining device 44 would continue to be under tension and be biased downward, for example, toward the bottom of the trash barrel 12 (FIG. 1). As the evaporation and/or shrinking of the air treating material progresses, retaining device 44 would remain under tension but decrease in the amount that it is biased toward the downward direction, for example, the bottom of the trash barrel 12 (FIG. 1) and return to its original at rest position (FIGS. 4, 6D).

The air treatment device 14 and specifically biased retaining device 44 is configured for keeping an air treating material 60 in place (even upon drying or shrinking thereof) when the cover of trash barrel 12, for example, a trash barrel with a built-in cover, removable cover, push in cover, built-in foot-pedal activated cover or other cover as is known in the art, is lifted or activated. Hence, the air treating material 60 is closely held in place and does not rattle, shake or move freely during use of the trash barrel 12.

Referring back to FIG. 2D, top portion 18 of air treatment device 14 includes air freshness indicator means 22 for use in indicating a measure of time that is commensurate with the depletion of the air treating material 60 housed within retaining device 44. More particularly, air freshness indicator means 22 includes a plurality of indicia 24, for example, the flower petals depicted in FIG. 2D, that change in appearance with a measure of time or with the use of multiple indicia 24 (as depicted in FIG. 2D) changes in appearance with various measures of time. For example, upon initial placement of air freshness indicator means 22 upon top portion 18, the indicia 24 may all be identical, that is, indicate one color or configuration. Upon some passage of time that is commensurate with a certain amount of depletion of the air treating material 60, one or more of the plurality of indicia 24 will change, for example, in color or configuration so as to indicate to a consumer that an amount of time or an amount of air treating material 60 has been depleted. Thus, the consumer will be notified by the indicia 24 that the air treating material 60 needs to be replaced. The air freshness indicator means 22 may also include a reset or on/off button 26 that may be used to reset or turn on or off the timing action of the air freshness indicator means 22. The air freshness indicator 22 may be of electronic, mechanical or chemical means and may be designed in the shape of a flower (e.g., with petals, with a stem and petals, with petals shaped like hearts, ovals or the like) and integrally molded or attachable through cooperating attachment means 54 such as, for example, hooks and loop fasteners, fastening clips, tape, adhesive, magnets, suction cups, screws, nails, other hardware and the like as is known in the art (FIG. 2E) to any location on the exterior of the air treatment device 14 or any cover. The air freshness indicator 22 is used to indicate to the user the depletion of the air treating material 60, not only as a function of quality of the air treating material but also as a function of time.

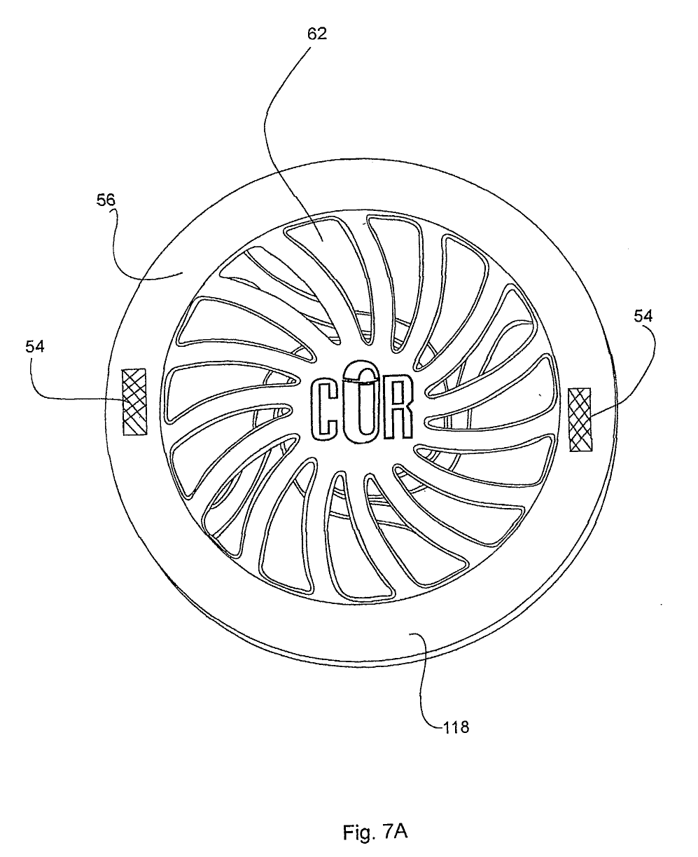

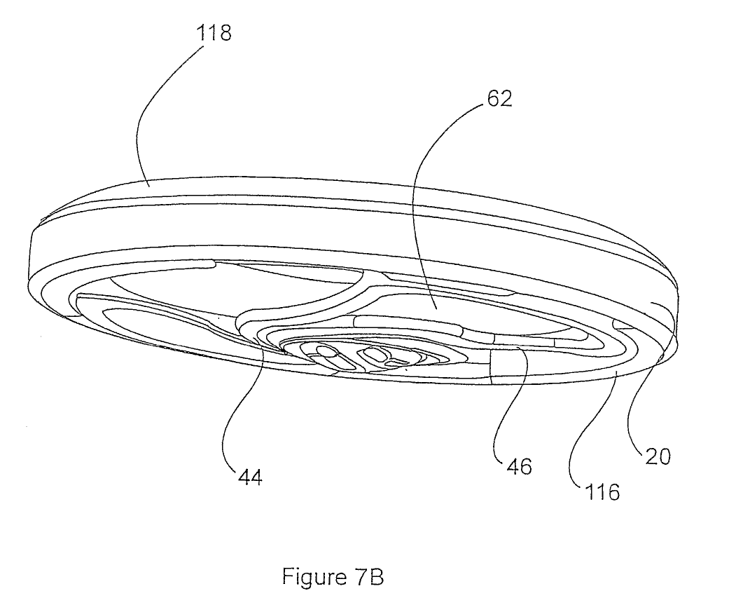

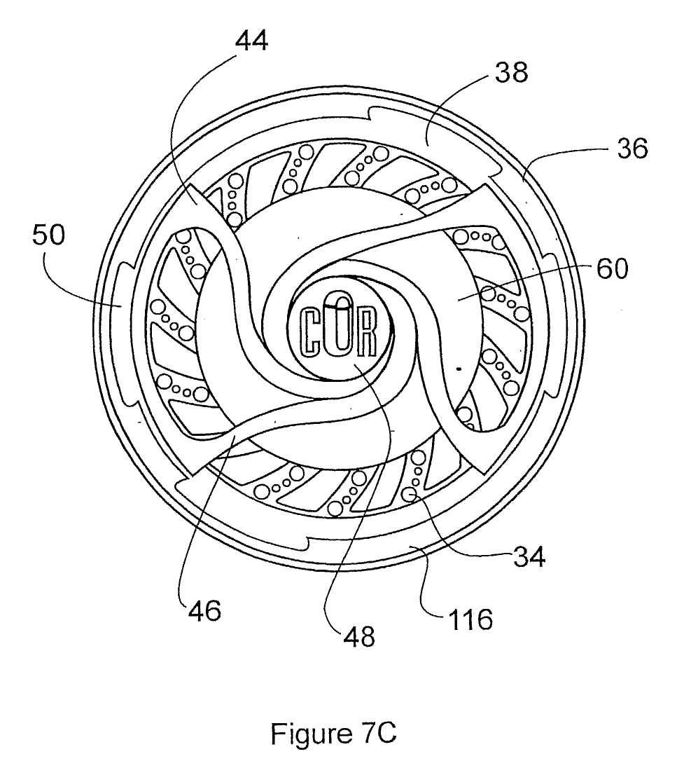

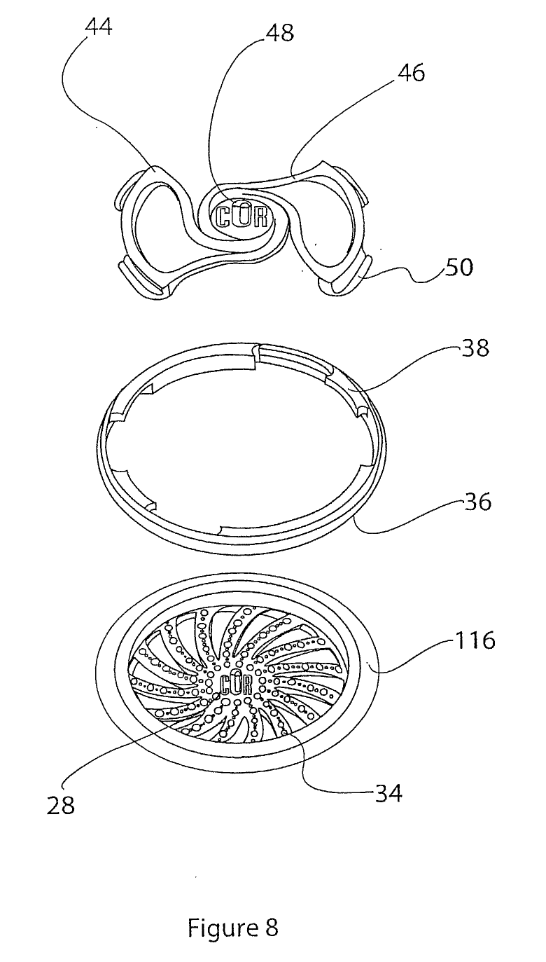

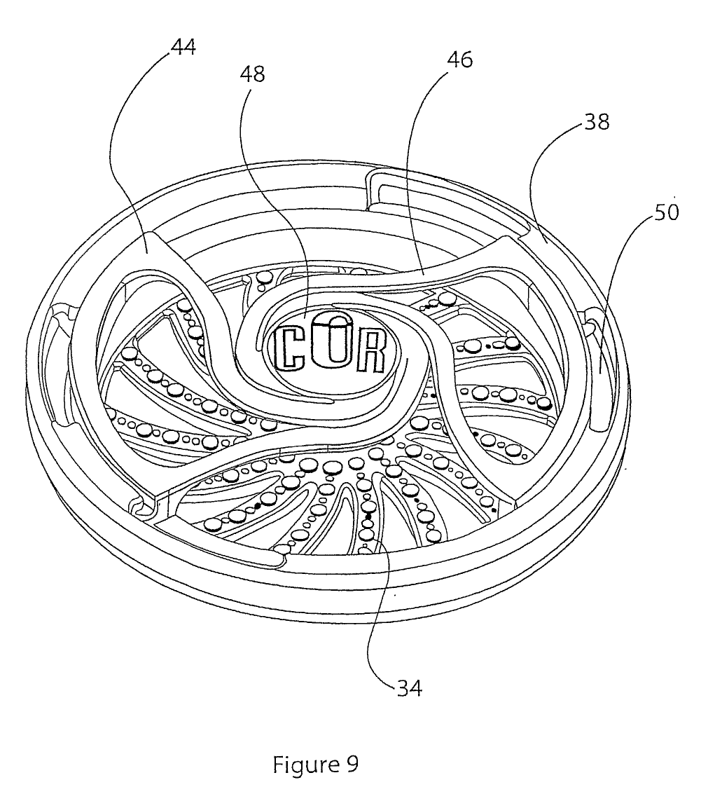

In an alternate embodiment and as shown in FIGS. 7A, 7B, 7C, 8, 9, 10C and 10D, wherein like reference numerals represent like components of the air treatment device 14, a separate or stand alone air treatment device 56 is disclosed. Stand alone air treatment device 56 is configured to attach to trash barrels 12 as an after market add on air treatment feature. The air treatment device 56 is substantially similar to the air treatment device 14 discussed herein. The air treatment device 56 further includes a stand alone housing having a top 118 portion (similar to a top cover and top portion 18) and bottom portion 116 (similar to a bottom cover and bottom portion 16). Similar to air treatment device 14, bottom portion 116 includes a retention ring 36 that may either be separate or integrally molded with bottom portion 116. Retention ring 36 includes retaining means 50 that cooperates with the removable or pivotal or hinged or clasped or otherwise retention of retaining device 44. The stand alone air treatment device 56 is configured to be attached to any cover or lid of a trash barrel 12 via attachment means 54. Air treatment device 56 is attached to the cover or lid member through cooperating attachment means 54 such as, for example, hook and loop fasteners, fastening clips, tape, adhesive, magnets, suction cups, screws, nails, other hardware and the like as is known in the art.

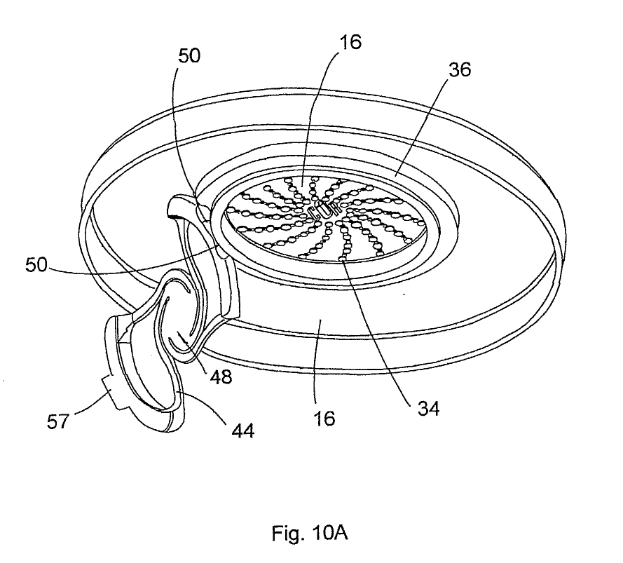

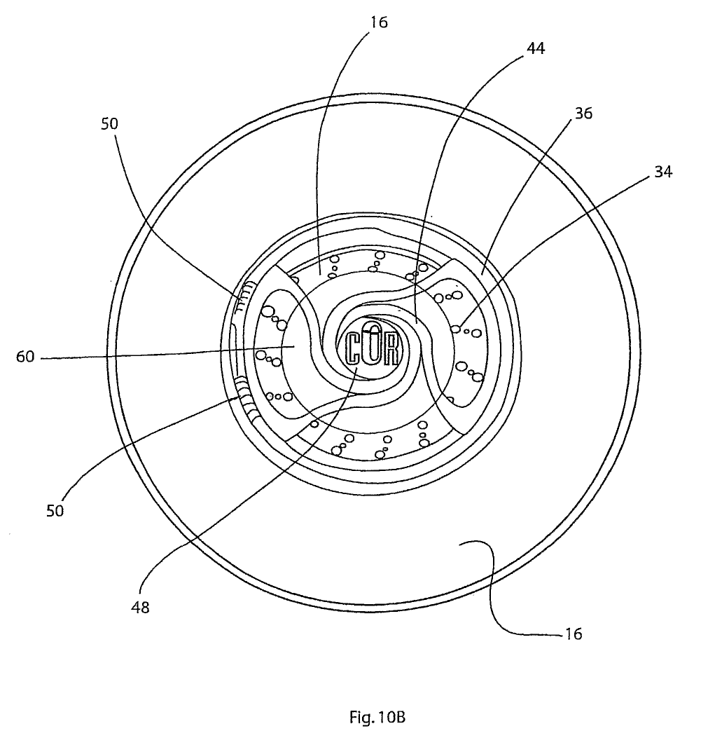

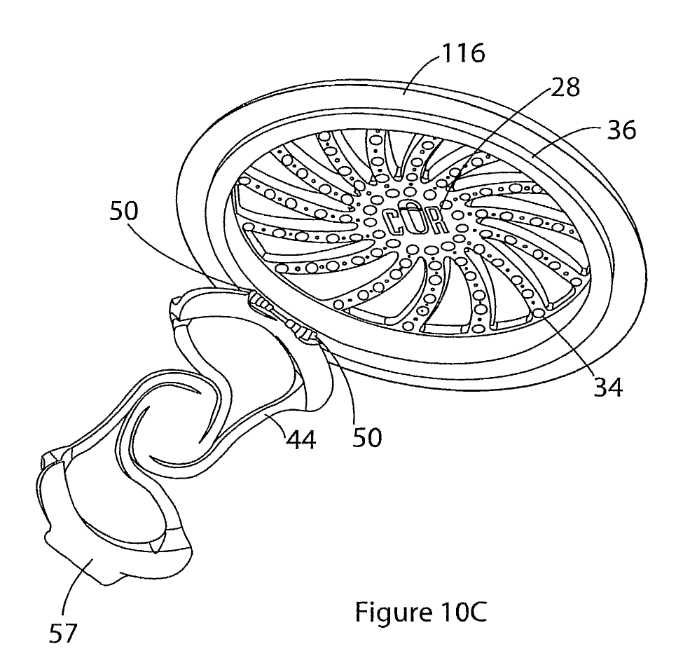

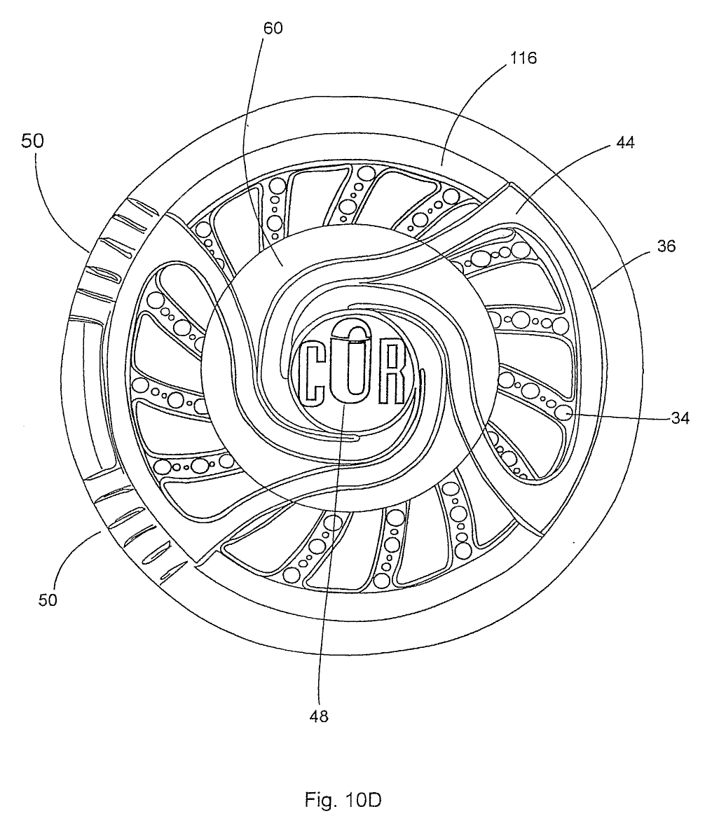

In yet another alternate embodiment and as shown in FIGS. 10A, 10B, 10C and 10D is the retaining device 44 which is pivotally coupled to the retention ring 36 via living hinges, spring biased hinges and the like as is known in the art for movement between the closed position (as depicted in FIGS. 10B and 10D) and the open position (as depicted in FIGS. 10A and 10C). A closure tab or detent 57 may be used for locking the retaining device 44 in place when the air treating material 60 is enclosed. Closure tab or detent 57 fits within a respective opening (not shown) of retention ring 36.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

1. A housing for use with a receptacle comprising:
a cover adapted for use with a receptacle; and
a housing member for housing an air treating material, the housing being removably coupled to the cover and is deformable with respect to the cover.

2. The housing according to claim 1, wherein the housing is biased toward the air treating material to thereby provide continuous contact of the air treating material and housing.

3. The housing according to claim 1, wherein the housing and the cover include an air treating material therebetween.

4. The housing according to claim 1, wherein the cover and housing include retaining means for removably coupling to one another.

5. The housing according to claim 4, wherein the housing is rotatably coupled to the cover.

6. The housing according to claim 1, further comprising an indicator means indicating a period of time.

7. The housing according to claim 6, wherein the indicator means is coupled to the cover.

8. An air treatment system comprising:
a housing for securing an air treating material;
a retaining means for removably coupling the housing to a receptacle, wherein the housing includes a biased portion for securely retaining the air treating material therein.

9. The air treatment system according to claim 8, wherein the biased portion retains a constant bias against the air treating material.

10. The air treatment system according to claim 8, wherein the housing is removably coupled to a cover portion of the receptacle.

11. The air treatment system according to claim 8, further comprising an indicating means for indicating a time frame.

12. The air treatment system according to claim 11, wherein the indicating means is coupled to the cover.

13. The air treatment system according to claim 8, further comprising a plurality of through holes for allowing air to contact the air treating material.

14. An air treatment device comprising:
a removably mountable basket; and
a cover coupled to the basket, wherein the basket is deformable with respect to the cover.

15. The air treatment device according to claim 14, wherein the basket is biased toward the cover.

16. The air treatment device according to claim 14, wherein the cover and basket include respective coupling members.

17. The air treatment device according to claim 16, wherein at least one of the coupling members includes a flange, thread, rail, detent, projection and tang.

18. The air treatment device according to claim 14, further comprising an attachment means for attaching the cover to a receptacle.

19. The air treatment device according to claim 18, wherein the attachment means includes hook and loop fasteners, fastening clips, tape, adhesive, magnets, suction cups, screws, and nails.

20. The air treatment device according to claim 14, further comprising a receptacle.

21. An air treatment system comprising:
a housing for securing an air treating material;
a retaining means for coupling the housing to a receptacle, wherein the housing includes a biased portion for securely retaining the air treating material therein.

22. A housing for use with a receptacle comprising:
a cover adapted for use with a receptacle; and
a housing member for housing an air treating material, the housing being coupled to the cover and is deformable with respect to the cover.

23. An air treatment device comprising:
a pivotally mountable basket; and
a cover coupled to the basket, wherein the basket is deformable with respect to the cover.

24. A device comprising:
a housing having an interior and exterior portion; and
a means for indicating a time period positioned on the housing.

25. A device according to claim 21, wherein at least a segment of the exterior portion translates with respect to the interior portion.

26. A device according to claim 22, wherein said housing contains any one of a foodstuff, beverage, insecticide, pesticide, air freshener, air deodorizer, odor absorber, antibacterial component, air treatment material, detergent, cleaning products, stain remover, pet litter, a filter component, fertilizer, toilet bowl cleaners, toilet bowl brush, tooth brush, drain clog remover, mildew remover, mold remover, soap scum remover, mop pad, sponge, automobile protectant, automobile washing products, automobile wax, automobile polish, leather protectant, carpet cleaner and upholstery cleaner.

27. An air treatment device comprising:
a housing having an interior and exterior portion, wherein the interior of the housing includes air treating material; and
a replacement indicator coupled to the housing.

28. The air treatment device according to claim 27, wherein the replacement indicator indicates a measure of time.

29. The air treatment device according to claim 28, wherein the measure of time is commensurate with the depletion or effective use of the air treating material.

30. The air treatment device according to claim 27, wherein the replacement indicator indicates a time to replace the air treatment device or air treating material.

31. The air treatment device according to claim 27, wherein the air treatment device further includes at least one suction cup.

32. The air treatment device according to claim 27, wherein the air treatment device treats odors within a refrigerator, freezer or a receptacle used to house food.

33. The air treatment device according to claim 27, wherein the air treatment material is baking soda.

34. The air treatment device according to claim 27, wherein the replacement indicator includes indicia.

35. The air treatment device according to claim 34, wherein the indicia is a color or change in color.

36. The air treatment device according to claim 27, wherein the housing includes a plurality of through holes for allowing air to contact the air treating material.

37. A device comprising:
a housing having an interior and exterior portion and air treatment material therebetween, the housing including a plurality of through holes for allowing air to contact the air treating material; and
an indicator attached to the device for indicating replacement of the device or the air treatment material.

38. The device according to claim 37, wherein the indicator indicates a measure of time.

39. The device according to claim 38, wherein the measure of time is commensurate with the depletion or effective use of the air treating material.

40. The device according to claim 37, wherein the device further includes at least one suction cup.

41. The device according to claim 37, wherein the device treats odors within a refrigerator or freezer.

42. The device according to claim 37, wherein the air treatment material is baking soda.

43. The device according to claim 37, wherein the indicator includes indicia.

44. The device according to claim 43, wherein the indicia is a color or change in color.
